(19) United States
(12) Patent Application Publication
(10) Pub. No.: US 2018/0299431 A1
(43) Pub. Date: Oct. 18, 2018

(54) Explant Assay to Determine the Potential for Compositions to Promote or to Inhibit Neural Tube Defects

(71) Applicants: Johannes Michael Salbaum, Baton Rouge, LA (US); Claudia Kappen, Baton Rouge, LA (US)

(72) Inventors: Johannes Michael Salbaum, Baton Rouge, LA (US); Claudia Kappen, Baton Rouge, LA (US)

(73) Assignee: Board of Supervisors of Louisiana State University and Agricultural and Mechanical College, Baton Rouge, LA (US)

(21) Appl. No.: 15/771,126

(22) PCT Filed: Oct. 26, 2016
(86) PCT No.: PCT/US16/58801

§ 371 Date: Apr. 27, 2018

Related U.S. Application Data
(60) Provisional application No. 62/247,424, filed on Oct. 28, 2015.

(51) Int. Cl. G01N 33/50

(52) U.S. Cl. CPC G01N 33/5088; G01N 33/5032

(57) ABSTRACT

An explant assay based on embryonic tissue from laboratory animals may be used to identify substances that promote neural tube defects—teratogens to be avoided in pregnancy, and substances that inhibit neural tube defects—supplements that might be taken with benefit during pregnancy—to help reduce the incidence of birth defects. The assay screens for compounds that improve or impede mesodermal cell migration from an explant containing the primitive streak and parts of the lateral mesodermal wings that have been dissected from the embryo of a mouse or other experimental animal. Impaired migration of nascent mesodermal cells in the primitive streak is the morphogenetic basis underlying the pathogenesis of neural tube defects. We also hypothesize that maternal diabetes adversely affects mesoderm during gastrulation, before neurulation occurs.

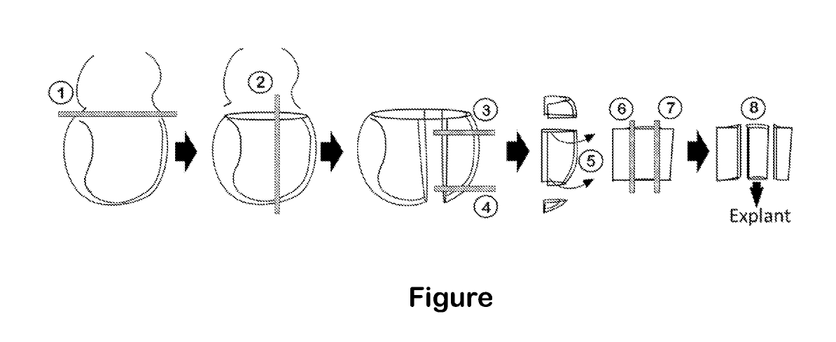

### PRIORITY CLAIM

The benefit of the 28 Oct. 2015 filing date of U.S. provisional patent application Ser. No. 62/247,424 is claimed under 35 U.S.C. § 119(e) in the United States, and is claimed under applicable treaties and conventions in all countries. The complete disclosure of the priority application is hereby incorporated by reference in its entirety.

### STATEMENT REGARDING FEDERALLY SPONSORED RESEARCH

This invention was made with government support under grant numbers RO1-HD085017, RO1-HD055528, and RO1-HD37804 awarded by the National Institutes of Health. The Government has certain rights in the invention.

### TECHNICAL FIELD

This invention pertains to assays to determine the effect of compositions in promoting or inhibiting neural tube defects.

### BACKGROUND ART

There is an unfilled need for an assay to identify substances that promote neural tube defects (teratogens to be avoided during pregnancy), and substances that inhibit neural tube defects (supplements that might be taken with benefit during pregnancy)—particularly in, but not limited to, pregnancies accompanied by maternal diabetes.

Birth defects caused by maternal diabetes present a significant burden to public health. Malformations of the heart, neural tube, and caudal region are up to ten times more frequent in these pregnancies. It has been a challenge to explain how such seemingly disparate phenotypes can all result from maternal diabetes.

Failure of the neural tube to close can produce birth defects whose severity ranges from spina bifida to lethal anencephaly. Environmental risk factors are thought to be important, such as maternal diabetes or folic acid deficiency. It is not well understood how altered maternal metabolism can interfere with embryonic development, particularly with neurulation.

Approximately 400 genes have been identified in the mouse for which mutations either cause or contribute to neurulation defects. Comparatively few genetic risk factors for neurulation defects are known in humans.

Despite improved dietary folic acid supply and better glycemic control, the incidence of neural tube defects (NTDs) has been reduced only partially. There is an unfilled need for a better understanding of how environmental factors interfere with embryonic development in general, particularly with neurulation. There is also an unfilled need for better assays to identify factors that can contribute to or inhibit neurulation defects, to better identify both teratogens and supplements to help reduce the incidence of birth defects.

J. Wallingford et al, “The continuing challenge of understanding and preventing neural tube defects.” Science, author manuscript, available in PMC 10 Jun. 2013, published in final edited form as Science 2013 Mar. 1; 339 (6123) provides a review of neural tube development and neural tube defects. The authors said, “In conclusion, NTD etiologies remain undefined, NTD population risk remains stubbornly high, and the occurrence of NTDs translates to a great cost in terms of physical, emotional, and financial burden placed on the affected child and their families.”

J. Salbaum et al., “Neural tube defect genes and maternal diabetes during pregnancy,” Birth Defects Res A Clin Mol Teratol, author manuscript, available in PMC 28 Nov. 2012, published in final edited form as Birth Defects Res A Clin Mol Teratol 2010 August; 88(8):601-611 reported that maternal diabetes profoundly affects gene expression in the developing embryo, in particular in a set of known NTD genes. In rodent experimental systems, NTDs present as phenotypes of incomplete penetrance in diabetic pregnancies. NTDs in diabetic pregnancies typically occur in only a fraction of exposed embryos, and those NTDs display wide differences in phenotype. This property is difficult to reconcile with observations of consistently altered gene expression in exposed embryos. Maternal diabetes was reported to increase the overall variability of gene expression levels in embryos. This report proposed that maternal diabetes reduces the precision of gene regulation in exposed individuals. Loss of precision in embryonic gene regulation could include changes to the epigenome via deregulated expression of chromatin-modifying factors.

C. Burdsai et al., “The role of E-cadherin and integrins in mesoderm differentiation and migration at the mammalian primitive streak,” Development 1993; 118:829-844 describe a system for the culture of mouse primitive streak tissues. Explants of epiblast or mesoderm tissue were dissected from the primitive streak of 7.5- to 7.8-day mouse embryos and cultured on a fibronectin substratum in serum-free, chemically defined medium. After 16-24 hours in culture, cells in explants of epiblast exhibited the typical close-packed morphology of epithelia, and the tissue remained as a coherent patch of cells that expressed transcripts of the cytokeratin Endo B. In contrast, cells in explants of primitive streak mesoderm exhibited a greatly flattened, fibroblastic morphology, did not express Endo B transcripts, and migrated away from the center of the explant.

### SUMMARY OF THE INVENTION

We have discovered an explant assay using particular embryonic tissues from mice or other laboratory animals to identify substances that promote neural tube defects (teratogens to be avoided in pregnancy), and substances that inhibit neural tube defects (supplements that might be taken with benefit during pregnancy, to help reduce the incidence of birth defects)—particularly in, but not limited to, pregnancies accompanied by maternal diabetes. The assay screens for compounds that improve or impede mesodermal cell migration from an explant containing the primitive streak and parts of the lateral mesodermal wings that have been dissected from the embryo of a mouse or other experimental animal.

We have discovered that impaired migration of nascent mesodermal cells in the primitive streak is the morphogenetic basis underlying the pathogenesis of neural tube defects. A related hypothesis is that maternal diabetes adversely affects mesoderm during gastrulation, before neurulation occurs. We have supporting evidence for our hypothesis, obtained from two independent mouse models of diabetic pregnancy. We conclude that perturbed gastrulation not only explains neurulation defects, but also provides a unifying etiology for a broad spectrum of congenital malformations in diabetic pregnancies. Gastrulation deficiencies represent a unifying mechanism that could explain all three types of birth defects arising from maternal diabetes: heart defects, neural tube defects, and caudal growth defects. (Incidentally, the validity of the novel explant assay does not require that our mesoderm/gastrulation hypothesis be correct.)

### BRIEF DESCRIPTION OF THE DRAWING

The FIGURE depicts schematically a dissection procedure for preparing an embryonic tissue explant to be used in an assay in accordance with this invention.

### MODES FOR CARRYING OUT THE INVENTION

We have observed in two independent mouse models of type I diabetes—namely, the Non-Obese Diabetic (NOD) strain of mice with spontaneously occurring diabetes, and the FVB strain of mice with chemically-induced diabetes—that a significant fraction of embryos from diabetic dams develop severe morphogenetic defects of the open neural plate, in the form of cellular protrusions. These protrusions emerge before—and may in fact prevent—neural tube closure. We hypothesize that gastrulation itself, prior to neurulation per se, is the step where maternal diabetes induces neural tube defects (NTDs).

We support our hypothesis with new observations from neurulation-stage mouse embryos, in which we observed maternal diabetes to induce ectopic protrusions in the neural plate. Such protrusions are not a novel observation per se, but to our knowledge maternal diabetes has never previously been implicated in causing such protrusions. We have found that protrusions in maternal diabetes-exposed embryos are not rare: they were detectable in 25% of embryos of diabetic dams of the Non-Obese Diabetic (NOD) strain of mice, and in 13% of embryos of FVB dams (on the Purina 5015 diet) with chemically-induced diabetes. We have also observed protrusions in diabetic pregnancies in C57B16 mice. The location and incidence of the protrusions correlated with the frequencies of NTDs in the trunk region in both NOD and FVB models. Protrusions were seen only in diabetic, but not in normoglycemic pregnancies, suggesting that the defects were not due to a particular genetic background, but rather were a direct consequence of maternal diabetes.

The notion that teratogenic effects of maternal diabetes arise from effects on gastrulation is not in itself new, but to our knowledge there has never been a previous suggestion for a plausible underlying mechanism. Our data now suggest that alteration of primitive streak gene expression and mesodermal cell migration can explain the three most common types of birth defects in diabetic pregnancies. Histological analyses have shown that protrusions represent an aberrant gastrulation process, leading to the accumulation of ectopic mesoderm cells. The newly-proposed mechanism readily explains why most neural tube defects are limited to a specific region of the neural tube, despite the systemic insults of maternal diabetes. Our data show that maternal blood glucose levels change embryonic gene expression, and suggest that these changes in expression increase the susceptibility of the embryo to a malformation. A specific trigger event then causes malformation in a regionally restricted area, leading to one of the typically observed NTD phenotypes.

The concept that the nascent mesoderm arising during gastrulation is sensitive to maternal diabetes is believed to be novel. The novel explant assay is based on a change in perspective, shifting from “fuel-mediated teratogenesis” to a developmental signaling mechanism. Without this shift in perspective, there would have been little motivation to propose the novel explant assay disclosed here. We have observed that specific patterns of gene expression are associated with the protrusions, which contain a mesodermal core. However, because detailed knowledge of the differential gene expression is not necessary to perform the assay disclosed here, those details are omitted here in the interest of brevity. Those details are described in priority application Ser. No. 62/247,424, the complete disclosure of which is hereby incorporated by reference.

The Non-Obese Diabetic (NOD) strain of mice, which is prone to spontaneously develop autoimmune diabetes, is an established model for human type I diabetes. Embryos of diabetic NOD pregnancies are afflicted with a very high rate of neural tube defects (NTDs) and heart defects, another hallmark of diabetes teratogenicity.

We found that in embryos from hyperglycemic NOD dams, ectopic tissue protruded from the neural plate in various locations along the anterior-posterior axis. Such protrusions were only seen in diabetic pregnancies, in which they occurred in 25.3% of embryos at 8.5 days of gestation (E8.5). To test whether protrusions were unique to the NOD strain, we induced hyperglycemia with Streptozotocin in female mice of the FVB strain, resulting in an NTD incidence of 21.6%. At E8.5, 12.9% of hyperglycemia-exposed FVB embryos displayed similar protrusions. The overall appearance, location along the anterior-posterior axis, and internal organization of the protrusions were strikingly similar to those from NOD embryos. Thus we concluded that the malformations were not a peculiarity of the NOD genetic background, but instead arose as a consequence of the severe maternal hyperglycemia common to both experimental models.

Imaging by two-photon confocal microscopy, three-dimensional reconstruction of whole embryos from optical sections, and subsequent generation of single-plane views allowed closer examination of the junctures between protrusions and the embryo. We observed contiguity between the protrusion and the neural plate, with the outer layer resembling neuroepithelium. The core of a protrusion had lower cell density, reminiscent of mesenchymal character. This observation suggested that protrusions are not composed exclusively of neuroepithelial cells.

To determine the origin of protrusions, we profiled gene expression on microdissected protrusion tissue from the FVB model, using 3′-expression tag sequencing. For comparison, we laser-microdissected open neural tube immediately anterior of closure site 1. Analysis of Noto gene expression indicated that the neural tube samples were free from potentially contaminating notochord tissue. We identified 799 genes with statistically significant differential expression in protrusions as compared to open neural tube, and confirmed the sequencing-based observations by quantitative RT-PCR for selected genes. Hierarchical clustering demonstrated a clear distinction between expression profiles for protrusions and open neural tube. Annotations for 570 genes with predominant expression in protrusions were significantly enriched for the GO terms “mesoderm formation” and “mesoderm development.”

Analyses of NOD embryos by in situ hybridization at E8.5 revealed parallels between protrusions arising from spontaneously-initiating- and chemically-induced-maternal hyperglycemia. For example, Sox2, a marker for the epiblast/neuronal lineage, was present throughout the outer layer of the protrusion, whereas T, a marker for primitive streak and nascent mesoderm, extended into the proximal core of the protrusion. Tbx6, a marker for committed mesoderm, was found in the primitive streak and also in migrating cells of the mesodermal wings. Tbx6 expression in the core of a protrusion was reminiscent of proper mesoderm development: Expression was strongest where T expression had already been extinguished. Overall, our data indicated that migration of mesodermal cells was not completely blocked, but rather was impaired locally around the site of the protrusion.

The ectopic mesoderm in the protrusions could have resulted from altered proliferation of newly generated mesodermal cells, or from disoriented migration of nascent mesoderm. Our histological analyses supported the second possibility: We found no evidence for increased cell proliferation; staining for the mitosis marker Phospho-Histone 3 did not show differences between the protrusions and the rest of the embryo. Instead, we detected deposition of laminin between mesodermal cells within and at the base of the protrusions. This deposition was paralleled by the protrusion-prevalent expression of Laminin α5 and Nidogen 2 (components of the basal lamina), and Integrin α6, a receptor for Lama5. These data implicate either altered cell adhesion, or impaired migration of mesodermal cells in the formation of protrusions.

Evidence for impaired migration came from explant cultures under conditions that supported migration and differentiation of mesoderm, confirmed by staining for Vimentin. In these cultures, outgrowth from posterior tissue explants from diabetes-exposed NOD embryos at E7.5 or E8.5 was significantly reduced as compared to migration from explants from embryos from normoglycemic NOD pregnancies. For diabetes-exposed embryos, we also compared cultures of dissected protrusions to explants of the adjacent posterior tissue at E8.5. Cells from the protrusions either failed to migrate from the explant, or migrated significantly less as compared to the outgrowth observed from the corresponding posterior tissue explant. The reduced migration of cells from protrusions could not be attributed to developmental immaturity, as cells from earlier embryos exhibited comparable migratory capacity in these assays. The extent of the outgrowths was also uncorrelated with the size of the starting explant. We therefore concluded that exposure to maternal diabetes was responsible for the impaired migratory capacity of mesoderm in protrusions. Finally, outgrowth of explants from diabetes-exposed NOD E8.5 embryos was reduced in comparison to that from explants from diabetes-exposed E8.5 embryos of the FVB strain, which could explain the higher incidence of protrusions in the NOD model as compared to that seen in FVB embryos.

Our observations also demonstrated that culture conditions, including supportive extracellular matrix and growth factors present in fetal calf serum, did not suffice to rescue the cell migration deficiencies during the 26 hours of explant culture. Culture at lower glucose concentrations had no effect on outcome (p=0.23 for E7.5, p=0.22 for E8.5). Thus the explant cultures supported our hypothesis that exposure to maternal diabetes in utero causes impaired cell migration, and reduces egress from the primitive streak, which in the most severely affected individuals produces protrusions from the neural plate.

Intriguingly, protrusions tended to form at discrete anterior-posterior locations, rather than along the entire primitive streak. Consistent with prior data generally, we found that two thirds of NTDs in NOD embryos (˜26% of all embryos) involved the trunk region; this proportion was comparable to a protrusion incidence of 25%, of which almost all appeared in the region covered by the primitive streak. In the FVB model, half the NTDs (˜11% of all embryos) involved the trunk region, a rate that also paralleled the protrusion incidence (12.9%). Thus in both diabetes models defective mesoderm migration accounted for the vast majority of trunk and caudal neural tube defects in mouse diabetic pregnancies. Protrusions in more anterior locations were detected only occasionally. Even within the primitive streak region, protrusions were generally limited to particular locations, possibly indicating a limited time window for perturbations that can contribute to the formation of protrusions.

There are at least three possibilities (not mutually exclusive) for how protrusions might cause neural tube defects: (i) by preventing formation of the medial hinge point required for initial bending of the neural plate, (ii) by compromising elevation and bending of future neuroepithelium due to diminished cell migration into the underlying mesoderm, and (iii) by physically interfering with the closure of the neural tube at the dorsal midline. Careful examinations and histological analyses of embryos with protrusions revealed properly closed neural tubes rostral to the protrusion, with neurulation failure caudal to the protrusion, suggesting that the protrusions physically interfered with closure of the neural tube.

We have identified impaired mesoderm migration as the principal morphogenetic factor underlying the pathogenesis of NTDs. Since these NTDs are associated with environmental risk factors such as maternal metabolic disease, our findings imply that mesoderm migration is sensitive to metabolic state. Mesoderm migration also appears to be responsive to composition of the maternal diet; we have previously demonstrated that diet modulates the rate of NTDs in the FVB model. In the NOD strain, NTD incidence was reduced by folinic acid supplementation, similar to the beneficial effects of folic acid seen in STZ-induced diabetic mouse pregnancies. These findings support the proposition that metabolic factors can affect mesoderm formation and migration, and help identify cellular and molecular targets for preventing neural tube-related birth defects.

The most common congenital malformations in human diabetic pregnancies are heart defects, neural tube defects, and caudal growth defects, all of which have been postulated to arise before the 7th week of pregnancy. Our results support the proposition that perturbed mesoderm migration during gastrulation is the common cause underlying these seemingly different birth defects: (i) neural tube defects arise as a consequence of impaired mesoderm migration; (ii) early heart progenitors originate and migrate from the primitive streak; and (iii) caudal growth defects are also consistent with altered mesoderm formation and migration in the posterior primitive streak. Similarly, mesodermal deficiencies are believed to underlie the vertebral, cardiac, renal and limb malformations of VACTERL and axial mesodermal dysplasia phenotypes, which have also been linked to maternal diabetes. Our discovery of aberrant mesoderm migration during gastrulation in two different mouse models of Type I diabetes provides a unifying cellular mechanism that can explain both the developmental timing and the morphogenetic origin of the most common structural anomalies in diabetic embryopathy.

### EXAMPLE 1

### A Preferred Embodiment of an Embryonic Tissue Explant Assay to Screen for Compounds that Improve or Impede Mesodermal Cell Migration

Sterile techniques were followed through all steps of the procedure. Mouse (Mus musculus) embryos were prepared from either diabetic or normoglycemic mothers at 7.5 days of gestation, equivalent to Theiler stage 11. See www[dot]emouseatlas[dot]org/Databases/Anatomy/Diagrams/ts11/. Embryos were dissected free of uterine and decidual tissues in sterile phosphate-buffered saline, and were transferred into OPTI-MEM medium supplemented with 5% fetal calf serum. Ectoplacental cone and parietal endoderm were removed with #55 forceps. A series of cuts with a fine-tip glass needle produced the embryonic tissue segment used in the explant assay. The FIGURE depicts schematically the steps in the explant preparation procedure.

Cut 1 removed extraembryonic tissues. Cuts 2 and 3 separated the anterior and posterior regions of the embryo. Cut 4 removed the lower section of the tissue fragment, where the curvature of the tissue would otherwise impede Cut 5. Cut 5 transformed the tissue into an “open book' configuration. Cuts 6 and 7 then trimmed the sides. The result was a piece of embryonic material (labeled 8 in the FIGURE) that comprised the primitive streak and portions of the lateral mesodermal wings. Embryonic material 8 is the preferred tissue to be used in the explant assay.

The explant assay measured the migration of newly-formed mesodermal cells away from the explant itself. Explant tissue 8 was transferred into one well of a twelve-well tissue culture plate coated with Matrigel (thin coat procedure). Each well contained 2 mL of medium (DMEM supplemented with 20% fetal calf serum, and 55 μM β-mercaptoethanol). Explants were incubated in a tissue culture incubator at 37° C. under a 5% CO₂ atmosphere for 6 hours to allow them to settle and adhere to the bottom of the plate. After 6 hours, the size of the explant, including any surrounding cells, was documented and measured by microphotography. Explants were returned to the incubator for an additional 20 hours to permit newly formed mesodermal cells to migrate from the explant. After 20 hours, the size of the explant, including any surrounding cells, was again documented and measured by microphotography. The ratio of the areas occupied by explant and any surrounding cells before and after the 20-hour incubation is the “migration index.”

The assay may be used to test the extent to which substances such as drugs, botanical extracts, nutritional supplements, or other compounds or compositions affect mesodermal cell migration from the explant; whether they improve or impair migration; whether they can potentially remedy the impaired migration of cells from maternal diabetes-exposed embryos; whether they act as teratogens, or whether they can potentially reduce the incidence of neural tube defects. For such testing, the growth medium for the explant is replaced after the initial 6-hour settling time, as otherwise described above, with medium (DMEM supplemented with 20% fetal calf serum and 55 μM β-mercaptoethanol) supplemented with the particular compound(s), drug(s), botanical extract(s), or other agent(s) to be tested. With the migration index as the output, the assay is used to determine efficacy and effective concentration range for each substance tested.

Substances that improve mesodermal cell migration from the explants are considered candidates for preventing or ameliorating the spectrum of birth defects known as “diabetic embryopathy.” Substances that impede mesodermal cell migration from explants are considered potential teratogens.

### EXAMPLE 2

### Additional Embodiments of Embryo Explant Assay

The explant assay, as otherwise described in Example 1 for mouse embryos, can also be carried out with embryos from other vertebrate laboratory animals, such as Gallus gallus domesticus (chicken), Xenopus laevis (African clawed frog), or Danio rerio (Zebrafish). The details of the dissection procedure are modified as appropriate to prepare a section of the embryo containing the posterior primitive streak and part of the mesodermal wings. The assay can also be used, with appropriate modifications, to test the migration of mesodermal cells from embryoid bodies obtained by aggregation of embryonic stem cells, epiblast stem cells, or induced pluripotential stem cells. As a further alternative, the assay can use other growth media otherwise known in the art. The migration index in each case is the ratio of surface area occupied when explants have settled (the initial 6 hours) to the ratio of surface area occupied by explant and migrating cells after a further 20 hours in culture. (These times, 6 hours and 20 hours, were chosen empirically and for convenience, and they may be modified to fit particular conditions or settings, 24 hours and 48 hours, for example, would also be useful time periods for the assay.) The migration index is used to judge whether a substance has beneficial or detrimental effects on mesodermal cell migration.

### Materials and Methods

### EXAMPLE 3

### Mice and Embryos

All animal experiments were performed with prior approval from the Pennington Biomedical Research Center IACUC, and were conducted in accordance with the “Guide for the care and use of laboratory animals” of the United States National Institutes of Health. Animals were housed with a 12-hour light/dark cycle, with access to food and water ad libitum. Mice of the Non-Obese Diabetic strain (NOD/ShiLtJ, The Jackson Laboratory, Bar Harbor, Me.; sometimes referred to as “NOD” mice), as well as male mice of strain FVB were kept on Purina™ 5001 diet (calories provided by: Protein, 28.5%; Fat, 13.5%; Carbohydrate, 58%. LabDiet, Purina Mills Inc., Gray Summit, Mo.). Females of strain FVB were kept on Purina 5015 diet (calories provided by: Protein, 19.8%; Fat, 25.3%; Carbohydrate, 54.8%). Blood glucose levels were monitored weekly in NOD mice. Once blood glucose exceeded 250 mg/dl, females were considered diabetic, and were then used for mating and production of embryos. Diabetic as well as normoglycemic NOD female mice were mated to normoglycemic NOD male mice; once NOD males became hyperglycemic, they were retired from the experiment and were no longer used.

Diabetes in FVB female mice was chemically induced with Streptozotocin (Sigma-Aldrich, St. Louis, Mo.) according to published protocols (Salbaum, J. M. et al. Altered gene expression and spongiotrophoblast differentiation in placenta from a mouse model of diabetes in pregnancy. Diabetologia 54, 1909-1920, doi:10.1007/s00125-011-2132-6 (2011)). Once blood glucose levels exceeded 250 mg/dl, mice were used for mating with normoglycemic FVB male mice. Noon of the day of the appearance of a vaginal mating plug was designated day 0.5 of gestation. Embryos were dissected from the uterus at 8.5 days of gestation using a Leica MZ6 stereomicroscope (Leica Microsystems, Buffalo Grove, Ill.). Extraembryonic endoderm and amnion were removed, and embryos were either fixed in 4% paraformaldehyde in phosphate-buffered saline (PBS) at 4° C., or placed in Tissue-Tek O.C.T. compound (Fisher Scientific, Pittsburgh, Pa.), frozen, and stored at −80° C.

### EXAMPLE 4

### Imaging of Embryos

Embryos were prepared for histological or molecular analysis at 8.5 days of gestation from hyperglycemic NOD or FVB dams, or from strain-matched normoglycemic control dams. Embryos with malformations were fixed, stained with DAPI (4′,6-diamidino-2-phenylindole), and imaged by 2-photon confocal microscopy.

Fixed embryos were stained in PBS/4% paraformaldehyde containing DAPI to help visualize cell nuclei. Embryos were washed in PBS, dehydrated in a graded alcohol series, cleared in BABB (a 1:2 mixture of benzyl alcohol and benzyl benzoate), and imaged on a Leica SP5 Confocal microscope using 2-photon technology. Optical sections were used to generate three-dimensional reconstructions of individual embryos using Imaris software (Bitplane Inc., South Windsor, Conn.). Computationally-generated embryo surfaces as well as virtual sections were used to evaluate the anatomy of dysmorphologies.

### EXAMPLE 5

### Tissue Preparations

For laser microdissections, embryos (with 7-9 somite pairs) embedded in O.C.T. were used to produce cryosections at 16 μm nominal thickness on a Microm HM560 cryostat. Sections were mounted on glass slides with a PEN (polyethylene naphthalate) membrane, dried at 50° C. for 30 minutes, and stored in a vacuum desiccator. Laser microdissection was performed with a Leica LMD 6000 laser microdissection system. In a set of serial sections from a single embryo, the site of neural tube closure (closure 1) was identified. At the anterior side of the closure, open neural tube tissue was microdissected and collected from the ten sections preceding the first section that showed a closed neural tube. All microdissected tissue segments from an individual embryo were collected into Trizol (Life Technologies, Grand Island, N.Y.) for RNA preparations. RNA was quantified on a Qbit fluorometer (Life Technologies, Grand Island, N.Y.), and stored at −80° C. Ectopic tissues from embryos showing morphogenetic deficiencies of the neural plate were dissected using sterile #55 forceps (Ted Pella Inc., Redding, Calif.). Care was taken not to penetrate the neural plate during removal of the ectopic material. Tissue was directly transferred to Trizol for RNA extraction. RNA was quantified on a Qbit fluorometer, and stored at −80° C.

### EXAMPLE 6

### Molecular Analyses

To determine the etiology and identity of protrusion tissue, we profiled gene expression by expression tag sequencing on an AB SOLiD™ 5500XL sequencer. Expression profiles were compared between protrusion tissue and open neural plate prepared by laser microdissection immediately anterior of neural tube closure site 1. Sequence reads were mapped (RefSeq RNA, mm9) using SOLiDSAGE™ to generate count data for each gene. Differential gene expression was determined using DESeq, with validation of selected genes by qPCR. In situ hybridizations and immunohistochemical analyses were performed on cryosections following standard protocols. The migratory capacities of cells in protrusions and posterior embryonic tissue were assessed in explant cultures using time-lapse video and phase contrast microscopy.

Gene expression profiling was performed by expression tag sequencing (SAGE) on an AB SOLiD 5500XL next-generation sequencing instrument using reagent kits from the manufacturer (Applied Biosystems, Foster City, Calif.). Sequence reads were aligned to mouse RefSeq transcripts (version mm9) as the reference, using SOLiDSAGE (Applied Biosystems) software. Only uniquely mapped sequence reads were counted to generate the expression count level for each respective RefSeq gene.

Sequencing libraries were constructed from 7 ng of laser-microdissected open neural tube material, which yielded an average of 10.2 million mapped reads (after allowing for quality control sampling). Sequencing libraries from protrusion material were generated from 12 ng of total RNA material, which yielded an average of 8.9 million mapped reads (after allowing for alignment and quality control). Differential expression between open neural tube tissue and protrusion tissue was analyzed using the R/Bioconductor program DESeq version 1.81. A gene was considered to be differentially expressed if the adjusted p-value (Benjamini & Hochberg procedure) for the respective comparison was below 0.1. Hierarchical clustering analyses were performed using MeV (Multi Experiment Viewer, www[dot]tm4[dot]org/mev/), using a “Manhattan distance” correlation metric. Pathway analyses and biological annotation were performed using DAVID bioinformatics software. Sequencing-based expression data were validated by qPCR using cDNA equivalent to 50 pg total RNA per reaction, 4 technical replicates per gene, and normalization versus Pole4 as internal control.

### EXAMPLE 7

### Histological Analyses

In situ hybridizations on cryosections were performed using digoxigenin-labelled antisense riboprobes as otherwise described in Salbaum, J. M. et al. Altered gene expression and spongiotrophoblast differentiation in placenta from a mouse model of diabetes in pregnancy. Diabetologia 54, 1909-1920, doi:10.1007/s00125-011-2132-6 (2011); and Salbaum, J. M. Punc, a novel mouse gene of the immunoglobulin superfamily, is expressed predominantly in the developing nervous system. Mech Dev 71, 201-204 (1998). Immunohistochemistry was performed as otherwise described in Salbaum, J. M., Kruger, C. & Kappen, C. Mutation at the folate receptor 4 locus modulates gene expression profiles in the mouse uterus in response to periconceptional folate supplementation. Biochim Biophys Acta 1832, 1653-1661, doi:10.1016/j.bbadis.2013.04.028 (2013). Cryosections were stained with antibodies against Laminin (Abcam, Cambridge, Mass.) and Phospho-Histone 3 (directly labeled with Alexa Fluor 488; Biolegend, San Diego, Calif.); Laminin was detected by indirect immunofluorescence using a goat-anti rabbit Alexa Fluor 594-labeled secondary antibody (Life Technologies, Grand Island, N.Y.). Sections were counterstained with Hoechst 33342 (Life Technologies, Grand Island, N.Y.). Fluorescence was recorded on an Everest digital microscopy workstation using Slidebook software (Intelligent Imaging Innovations, Inc., Denver, Colo.).

### EXAMPLE 8

### Explant Cultures

Protrusions were microdissected from diabetes-exposed NOD embryos. Posterior tissues containing open neural plate, mesoderm, and underlying endoderm were dissected from normal or diabetes-exposed NOD or FVB embryos using glass needles. The explants were placed into 12-well tissue culture plates coated with Matrigel, and were then cultured in DMEM containing 20% FCS and 2-mercaptoethanol. Images were taken 6 hours after initiation of the cultures, and again 20 hours later, after a total of 26 hours in culture. At this time, explants from normal E7.5 NOD embryos are in a phase of active outgrowth (which continues for at least another 3 days without obvious signs of cell death), with the margin of the explant extending outward at an average speed of 0.32 μm/min. (±0.12 μm/min., n=10), as determined from time-lapse videos, using the Leica TIRF DMI6000 system.

These conditions support cell migration and differentiation of mesoderm, including cardiac mesoderm, as evidenced by the presence of rhythmically contractile areas in 7 of 14 outgrowths from primitive streak explants of E7.5 NOD embryos from normal pregnancies after 26 hours in culture. Outgrowth from an explant was expressed as distance migrated by the margin of the explant: For each image, the size of the area covered by the explant was determined, and the radius was calculated for a circle of the same area. The radius at the 6 h time point of the explant culture was subtracted from the radius for the area covered by the same explant at the 26 h time point of culture. Net outgrowth of explants from E8.5 diabetes-exposed NOD embryos over the 20-hour period was significantly slower (p=6.7×10⁻⁴), at an average of 0.27 μm/minute (±0.10 μm/min., n=39), than outgrowth for explants from normal E8.5 NOD embryos, at 0.44 μm/minute (±0.09 μm/min., n=8). Outgrowth over 20 hours from explants isolated at E7.5 was also significantly slower (p=0.002) for diabetes-exposed embryos, at 0.15 μm/minute (±0.09 μm/min., n=10), than outgrowth for normal embryos, at 0.32 μm/minute (±0.12 μm/min., n=10).

Other metrics were tested as well. The metric that gave the most useful results was the “difference in radius” metric described above, or equivalently, a metric proportional to the difference between the square roots of the two areas. (The area measured in each case is that of the entire explant plus any outgrowth.)

Explants were fixed in 4% paraformaldehyde, washed with PBS, incubated for 20 minutes in TBST, and blocked for one hour in PBS/10% goat serum/1% BSA. For staining, explants were incubated with Alexa488-labeled anti-Vimentin antibody, as well as Alexa594-labeled Phalloidin for 4 hours at room temperature, washed with PBS, incubated with PBS containing DRAQS, washed again with PBS and cover-slipped under Fluoromount G Fluorescence was recorded on an Everest digital microscopy workstation.

Note: Photographs typically do not reproduce well in patent publications, and photographs—although they can often be helpful for illustrative purposes—are not considered necessary to describe the operation of the present invention. Therefore, with the exception of the single FIGURE, other figures and photographs related to the disclosure are not reproduced here. Related photographs and figures may be viewed in the file of the priority application, U.S. provisional patent application 62/247,424; and in a related publication J. M. Salbaum et al., Novel Mode of Defective Neural Tube Closure in the Non-Obese Diabetic (NOD) Mouse Strain. Sci Rep. 2015 Nov. 23; 5:16917. doi: 10.1038/srep16917, including the Supplemental Materials that are available online; all of which are hereby incorporated by reference in their entirety.

### Results

### EXAMPLE 9

### Observations of Neural Plate Protrusion Phenotype in Embryos from Diabetic Pregnancies

In embryos from NOD strain diabetic pregnancies, neural plate protrusions were most commonly seen in caudal locations. In some embryos, protrusions were also seen at the hind brain level, rostral to the neural tube closure front; at the mid/forebrain region; and in the trunk area. Compared to unaffected regions, the areas affected by a protrusion displayed an overall normal organization, with the exception of a bulge at the midline. The bulge had an outer layer similar to and contiguous with the neuroepithelium, and a core in which cell nuclei were sparse. Embryos from diabetic pregnancies of the FVB strain displayed protrusions similar to those observed in the NOD strain.

### EXAMPLE 10

### Protrusions Featured Internal Deposition of Laminin

We observed that all protrusions, from early stage to mature stage, appeared to have internal accumulations of laminin in the mesenchyme. Additionally, the outer cell layer was typically separated from the internal mesenchyme by a laminin-positive layer of extracellular matrix.

### EXAMPLE 11

### Exposure to Maternal Diabetes Decreased Cell Migration in Explant Cultures

Outgrowth from an explant was quantified as the net outward distance migrated by cells at the margin of an explant between hours 6 and 26 after initiation of culture. We observed that posterior tissue explants from diabetes-exposed NOD embryos displayed significantly reduced outgrowth compared to explants from normal pregnancies at E7.5 (normal n=10, exposed n=7; p=1.7×10⁻³; 96.7% power at alpha=0.05) and at E8.5 (normal n=8, exposed n=39; p=6.7×10⁻⁴; 99.7% power at alpha=0.05). Explants of dissected protrusions produced significantly less outgrowth than the corresponding posterior tissue from the same embryo at E8.5 (n=12 pairs; p=6.2×10⁻⁵; 99.9% power at alpha=0.05). Posterior tissue explants from diabetes-exposed E8.5 NOD embryos (n=39) display reduced outgrowth compared to explants from diabetes-exposed FVB embryos at E8.5 (n=6; p=4.4×10⁻⁷; ˜100% power at alpha=0.05).

### EXAMPLE 12

### Effect of Protrusions on Neural Tube Closure

At 9.5 days of gestation we observed embryos with protrusions in various locations, for example: emerging from underneath the dorsal roof of the neural tube, preventing closure towards the caudal end of the embryo; or a long protrusion emerging at approximately the hind limb bud level, keeping the neural plate caudal to the protrusion open; or a bifurcated protrusion emanating from the neural tube at the level of the hind limb bud, keeping the neural tube open caudal to the point of emergence.

At 10.5 days of gestation, with forebrain and midbrain closed, we observed various neurulation failures and protrusions, including for example: neurulation failure caudal to the midbrain-hindbrain junction; or a small protrusion near the hindbrain—spinal cord junction; or a very small protrusion at the level of the forelimb bud, with neurulation completed successfully along the neuraxis except for the small area of the protrusion; or a larger protrusion slightly caudal to the forelimb bud, with the neural tube closed rostral to the protrusion while remaining open caudally from the protrusion to the end of the neuraxis.

### EXAMPLE 13

### NOD Diabetic Pregnancies and Folinic Acid

Supplementation with folinic acid (12.5 mg/kg body weight, once per week by intraperitoneal injection) was initiated at the age of 10 weeks in 56 NOD females. The folinic acid did not significantly affect the time to subsequent onset of hyperglycemia, as compared to unsupplemented NOD females (n=196) (Kaplan-Meier statistical test, p=0.8639). Maternal hyperglycemia levels at mating and at dissection were not significantly different between supplemented and non-supplemented groups (ANOVA; p=0.51). However, folinic acid supplementation reduced the incidence of neural tube defects in embryos (gestation day 10.5) in diabetic NOD pregnancies from 40.2% (39 NTDs in 97 embryos) to 21% (17 NTDs in 81 embryos) (Fisher's Exact Test, p=0.006).

The complete disclosures of all references cited in this specification are hereby incorporated by reference, as is the complete disclosure of priority application 62/247,424. Also incorporated by reference is the complete disclosure of the following post-priority date publication by the inventors and their colleagues: J. Salbaum et al., “Novel mode of defective neural tube closure in the non-obese diabetic (NOD) mouse strain,” Scientific Reports 2015; 5:16917. In the event of an otherwise irreconcilable conflict, however, the present specification shall control.

What is claimed:

1. A process for assaying the propensity of a composition to induce or to inhibit neural tube defects during vertebrate embryogenesis; said process comprising the steps of:
(a) dissecting an explant from a non-human vertebrate embryo at gastrulation stage, wherein said explant consists essentially of the primitive streak and parts of the lateral mesodermal wings of the embryo;
(b) culturing the explant ex vivo under conditions that are favorable for growth;
(c) adding the composition to the explant culture; and thereafter incubating the explant culture with the composition under conditions that are otherwise favorable for growth;
(d) measuring net outgrowth from the explant after said incubating step has continued for a period of time;
whereby: net outgrowth that is significantly greater than control (p<0.05) indicates that the composition has a tendency to inhibit neural tube defects; net outgrowth that is significantly less than control indicates that the composition has a tendency to induce neural tube defects; and net outgrowth that does not differ significantly from control indicates that the composition is expected to be neutral regarding the occurrence of neural tube defects.

2. The process of claim 1, wherein the non-human vertebrate embryo is a mammalian embryo, and wherein said dissecting step comprises:
(a) dissecting the embryo free from extraembryonic tissues;
(b) separating the posterior region of the embryo from the anterior region of the embryo;
(c) removing portions of the posterior region of the embryo, where the removed portions would otherwise impede step (d) were those portions not removed;
(d) unfolding the remaining portions of the posterior region of the embryo; and
(e) trimming away sides from the unfolded configuration to leave an explant consisting essentially of the primitive streak and parts of the lateral mesodermal wings of the embryo.

3. The process of claim 2, wherein at least one of steps (b), (c), (d), or (e) is carried out with a fine, drawn-out glass needle.

4. The process of claim 1, wherein the non-human vertebrate embryo is a mouse embryo (Mus musculus).

5. The process of claim 1, wherein the non-human vertebrate embryo is an African clawed frog embryo (Xenopus laevis).

6. The process of claim 1, wherein the non-human vertebrate embryo is a zebrafish embryo (Danio rerio).

7. The process of claim 1, wherein said assaying determines that the composition has a tendency to induce neural tube defects; and additionally comprising the step of advising women who are pregnant or who may become pregnant to avoid the composition as being a potential teratogen.

8. The process of claim 1, wherein said assaying determines that the composition has a tendency to inhibit neural tube defects; and additionally comprising the step of advising women who are pregnant or who may become pregnant to consume the composition, as being potentially beneficial in reducing the incidence of neural tube defects.

9. The process of claim 1, wherein the net outgrowth is quantified as a metric that is proportional to difference between:
the square root of the total area of the explant, together with any outgrowth, after said incubating step
minus
the square root of the total area of the explant, together with any outgrowth, before said incubating step.
